(19) United States
(12) Patent Application Publication
(10) Pub. No.: US 2009/0270919 A1
(43) Pub. Date: Oct. 29, 2009

(54) INTERSPINOUS DEVICE

(75) Inventor: Geraldo Marins Dos Reis, JR., Campinas (BR)

Correspondence Address: OBLON, SPIVAK, MCCLELLAND MAIER & NEUSTADT, L.L.P., 1940 DUKE STREET, ALEXANDRIA, VA, 22314, US

(73) Assignee: GM DOS REIS JUNIOR, Sao Paulo (BR)

(21) Appl. No.: 12/404,712

(22) Filed: Mar. 16, 2009

(30) Foreign Application Priority Data
Apr. 25, 2008 (BR) PI 080 1855-3

(51) Int. Cl. A61B 17/70

(52) U.S. Cl. 606/249

(57) ABSTRACT

The present invention discloses an interspinous device that enables the suitable and safe positioning of the spinous processes in column treatments. More specifically, it discloses a device constituted of a structure that facilitates the insertion thereof into patients, demanding less effort from surgeons and, principally reducing the size of the incisions needed in patients, being made of polymeric material and comprised by a central body (1) having wings (2) provided with at least one hole to fix the spinous processes. Additionally, said central body has bumps that assist with accommodating the device in spinous processes, and means to assist the handling of the device during surgeries.

### FIELD OF THE INVENTION

The present invention refers to an interspinous device for fixing in spinous processes of the vertebral column of a patient with spinal problems. More particularly, the present invention refers to a surgical rear fix implant, between spinal apophysis of the vertebrae in treatments for decompression and redistribution of axial load on the intervertebral disks.

The interspinous device that is the object of the present invention comprises structural characteristics that allow a better distribution of load on the patent's backbone, so as to increase the amplitude of the spinal canal and the exit canal of the column nerves. Additionally, it enables the suitable repositioning of the vertebrae of an individual suffering from compression and degeneration problems of the vertebral disks. The implant requires rapid, simple, efficient and agile surgery, by means of a small incision that is less aggressive to patients, since there is no need to separate the bone musculature from the backbone.

### BACKGROUND OF THE INVENTION

As known to persons skilled in the art, a large section of the population suffers from back pain due to a series of factors, including bad posture, sedentary habits, and incorrect positions during work time and/or household tasks. These vices of posture and efforts to which the vertebral column is submitted, over the years, ends up causing lesions and traumas in an individual's vertebral structure, as well as a significant and painful degenerative process of the vertebral column.

These lesions are the result of degeneration, compression and/or fracture of the intervertebral disks, and can be treated by introducing interspinous implants, which can maintain the stability of the vertebral column to increase the size of the spinal canal and allow the patient to have normal mobility, which is limited to the final degrees of extension of the column only.

Surgical treatments are increasingly common among the population, because over time, an individual's bone structure may sustain wear, most frequently in the joint regions, such as in the vertebral column, since it is responsible for keeping the human body in the upright position. Thus, over the years, and depending on the individual's lifestyle, pains and troubles arise in the vertebral column region when performing certain movements, e specially when such movements demand effort from the backbone.

Some kinds of devices, called intervertebral or interspinous implants, are known. They seek to solve the problems of patients suffering from certain spinal traumas or lesions, particularly in cases of wear, degeneration and compression of the vertebrae and particularly of the vertebral disks. These devices present various arrangements, formats and particular technical aspects, both relating to the manufacturing material and the ways of accommodating and fixing in spinous processes.

For example, it is possible to mention some models that comprise a device arranged between two spinous processes and is fixed by polystyrene tapes, which enclose the respective spinous processes in treatment.

However, as persons skilled in the art will appreciate, this kind of device has complex assembly, principally concerning the fixing method in the patient's spinous processes. More specifically, the use of this kind of device requires a considerably large incision, as the surgeon needs to have integral access to the spinous processes in order to be able to enclose them in polystyrene tape.

Further, there are devices that, despite eliminating the fixing tapes, are made of multiple parts, which can be attached together during surgery. More specifically, it is possible to note the complexity and difficulty encountered by doctors when implanting this kind of device. Additionally, they are made of metals, normally noble metal.

The state of the art, described in patent U.S. Pat. No. 5.645.599, also comprises a device formed by a single titanium part comprised of a central U-shaped body having pairs of arms that are inserted into the spinous processes of the vertebrae in treatment. However, this kind of device has certain drawbacks related to efficiency, functionality, safety and cost.

More specifically, due to its open arrangement, that is, U-shaped, its structure becomes more flexible, thus generating a spring effect when submitted to certain forces by movement of the patient's vertebral column. In many cases, this spring effect is not recommended, particularly in certain decompression treatments of the vertebral disks.

Additionally, over time, and depending on the patient's efforts and movements, the tendency is that the free ends of the U-shaped body will join, losing their functionality entirely. The joining of the free ends of said body results in the need for a new surgery, which is not convenient for patients.

In order to avoid this kind of damage, there is also provided an arrangement of a kind of cushion made of elastic material to sustain the movements of the vertebral column. However, it is noted that in this case there is an arrangement and contact between two kinds of different materials, over time this may wear the materials due to attrition.

Additionally, due to the structural arrangement of this cushion, it is necessary to increase the incision in patients to allow handling and accommodation of the cushion in the implant. As persons skilled in the art will appreciate, the use of separate parts increases the interface between the device and the patient's bones, and also increases the risks of migration of one of the parts. All this ends up implying in damage that can only be corrected by new surgeries.

Additionally, the pairs of arms of the device of the state of the art, responsible for accommodating and anchoring of the spinous processes, comprise a smooth, internal contact surface, which requires a considerable quantity of additional fixing means, of the screw kind or metallic cerclages to maintain the correct positioning of the device.

Another drawback of the devices of the state of the art is that none of them comprises adequate means to facilitate handling during implant in the patients, which requires the use of many surgical and auxiliary instruments and during surgeries. This renders the surgeries and surgical treatments more complex.

Nevertheless, as can be seen above, the devices of the state of the art are made of metallic material, particularly noble metals such as titanium due to its compatibility and acceptance by the human body. As known by persons skilled in the art, these metals are radiopaque that in clinical exams present radiological artifacts, also known as smudges in the region where they are found. Logically, this makes the clinical and radiological examination imprecise and often inconclusive, because doctors are unable to evaluate the real status of the vertebral segment(s) in treatment.

In view of the above, it is clear that interspinous implants that can be manufactured and implanted in patients in a simple and effective manner are not known. Additionally, due to the complexity and inefficiency, treatments with the devices of the state of the art are unfeasible for the vast majority of the population, principally for low-income countries, because often a series of surgeries are needed to ensure the patient's health.

### SUMMARY OF THE INVENTION

Therefore, with the aim of overcoming the deficiencies and solving the drawbacks referred to above, identified in the devices of the state of the art, the interspinous device that is the object of the present invention was developed.

More specifically, it is an object of the present invention to provide an interspinous device that allows the suitable and safe placement of the vertebrae of a patient with spinal problems. Additionally, it comprises an arrangement that adjusts to the anatomy of the spinous processes in a safe manner.

Further, another objective of the present invention is an interspinous device that has a structure that facilitates the insertion thereof into patients, requiring less effort from surgeons and, principally, reducing the size of the incisions required in patients.

Another objective of the present invention is an interspinous device made of polymeric material, which normally have radiolucent properties, that is, it does not interfere in radiological treatments.

The present invention also refers to interspinous devices that comprise means that assist handling thereof during surgeries.

To achieve the objectives proposed above, the interspinous device that is the object of the present invention is made of polymeric material, and formed by a central body having anchoring wings of the spinous processes, the inner surfaces of which are provided with protrusions. Said central body also comprises contact surfaces with bumps that assist in accommodating the device in the spinous processes. Additionally, the interspinous device has grooved holes in the rear region to encase and handle the surgical instruments, as well as means for detecting the correct placement of the device in radiological treatments.

### BRIEF DESCRIPTION OF THE DRAWINGS

The objectives, improvements and technical effects provided by 25 the interspinous device that is the object of the present invention will be apparent to persons skilled in the art after the following detailed description, which draws references to the schematic figures appended hereto, wherein:

FIG. 1 illustrates a front perspective view of the interspinous device according to the present invention;

FIG. 2 illustrates a rear perspective view of the device illustrated in FIG. 1;

FIG. 3 shows a side view of the device illustrated in FIGS. 1 and 2.

### DETAILED DESCRIPTION OF THE INVENTION

According to the figures mentioned above, the interspinous device that is the object of the present invention comprises a central body (1) having two pairs of wings (2), each pair being provided with at least one through hole (3) for anchoring in the spinous processes by way of suture, metallic cerciage, screws or any other equivalent means in the field of surgery.

It is important to point out that the quantity of through holes arranged on the wings (2) may vary according to the kind of fixing mechanism or interests of the surgeon, since it is possible to anchor the device of the present invention, in an embodiment in which only one of the wings of each pair of wings (2) is provided with a through hole (3). In other embodiments, also addressed by the invention, it is possible for all the wings to be provided with at least one hole (3).

Said at least one hole (3) may comprise different formats, for example: circular, rectangular, triangular, oval, or the device may comprise holes with different formats, that is, a pair of wings (2) comprises a circular hole (3), and the other pair of wings (2) comprises a rectangular hole.

Additionally, said central body (1) comprises a closed tubular structure having an opening (4).

It must be made clear that the term “closed”, used to define the central body (1) refers to a continuous structure, that is, there are no openings or free ends that might generate a spring effect or cause the collapse of the structure of the central body (1), as occurs with certain devices of the state of the art. Additionally, after implant of the present invention in the patient, the patent may carry out his activities and movements normally without running the risk of causing the closure of the central body (1).

Said central body (1) has a tubular section, particularly flattened and oblong in shape. Further, according to a preferred embodiment of the present invention, the front region of the central body (1) is provided with bumps (5) the purpose of which is to incase the device of the present invention safely in the upper and lower portions of the spinous processes in treatment.

Said bumps (5) comprise, preferably, a pointed triangular shape, also referred to in the art as clamps. The purpose of these clamps is to grasp part of the individual's bone to avoid the displacement of the device. More particularly, said main body (1) comprises at least one bump adjacent to each pair of wings (2), and may advantageously have a plurality, depending on the region of the column in which the device is implanted.

The internal surfaces of said wings (2) also have protrusions (6) that increase the fixing level between the device and the spinous processes. Said protrusions may be distributed so as to partially or wholly occupy the internal surface of the wings (2), which will depend on the characteristics of the spinous processes and the fixing level desired by the surgeon.

Hence, fixing and maintaining the interspinous device of the present invention in spinous processes of a patient are ensured by the combination of fixing elements used in through holes (3), the clamps (5) of the central body, and the protrusions (6) of the wings (2). The result of the combination of these means to incase and fix the device eliminates the risks of displacement of the device in any direction, consequently making the patient's treatment safer, more efficient and long-lasting.

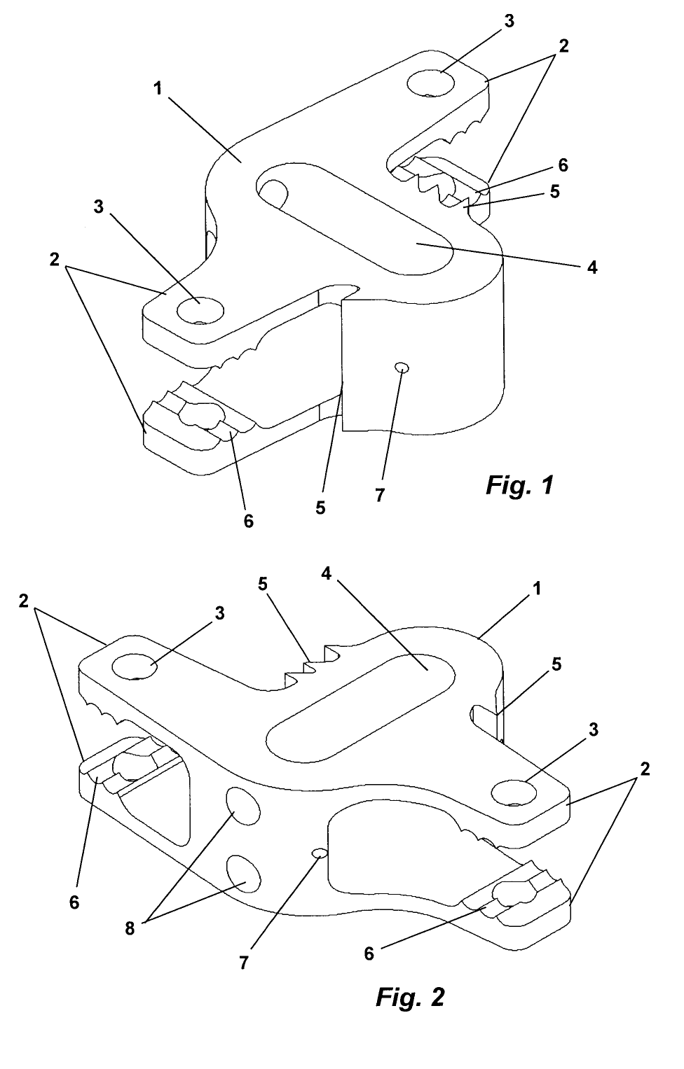

As can be noted in FIGS. 1 and 2, the front and rear regions of the central body (1) have a small canal (7) the purpose of which is to accommodate a metallic pin (not illustrated) of small diameter. Said canals (7) are rectilinear and perpendicular such that the metallic pins incorporated therein serve as positioning parameter, such that during radiological exams, doctors can precisely determine the place where the device is and, consequently, determine if undesirable displacement of the device has occurred.

Particularly in relation to FIG. 2, the central body (1) of the interspinous device that is the object of the present invention may advantageously comprise means to assist the handling of the device during surgeries. Said assisting means comprise at least one hole (8), and more advantageously two holes (8) on the inner wall which is grooved. The purpose of this hole (8) is to accommodate a surgical instrument, of the pincer kind, so that the surgeon can handle the device simply and perpendicularly in relation to the center of the body (1).

Therefore, with the arrangement of these means to assist the handling of the interspinous device, the incision in the region of the patient's vertebral column can be small, thus decreasing surgery time and a patent's post-operation recovery.

More particularly, the metallic pins incorporated in the canals (7) are useful when the interspinous device of the present invention is made of a polymeric material, and PEEK—Poly (ether-ether-ketone) is advantageously used, but could also be derivates thereof, such as, for example: PAEK, PEEKK, PEKK and PEK. Hence, due to the properties of this kind of material, that is, being radiolucent, in radiological exams the device is not detected, and said metallic pins act as an indicator so that doctors can precisely determine the position of the implant.

Although the present invention is more beneficial when made of polymeric materials due to the benefits highlighted above, it should be made clear that other materials could also be used, including some metals that area already used for surgical implants, for example, titanium.

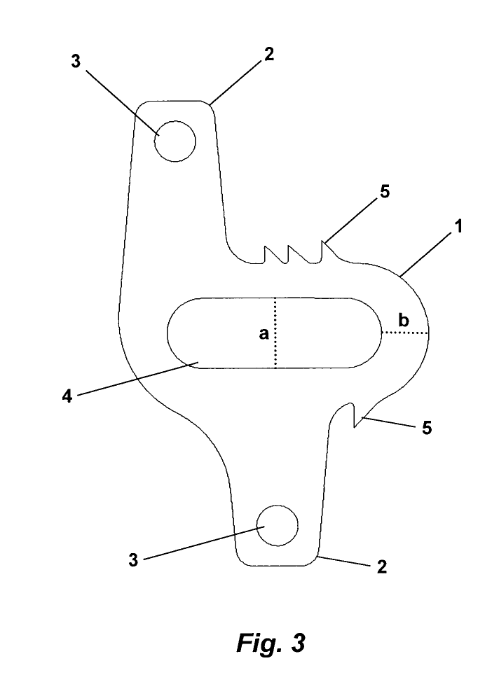

FIG. 3 shows a side view of the interspinous device, such that it is possible to see that the pairs of wings (2) are arranged in skewed fashion. In this figure, it is possible to define that the upper pair of wings is displaced towards the right, and the pair of wings inferior is relatively centralized. This displacement between the said pairs of wings allows the incasing and fixing of a series of devices along a sequence of spinous processes, even those adjacent to one another.

In a preferred embodiment of the interspinous device of the present invention one of said pairs of wings (2) is arranged relatively in the center and the other aligned with the rear portion of the central body (1).

Despite defining the term upper and lower in the paragraph above, the interspinous device can be inserted into vertebral column of a patient by inverting said pairs, which will depend on the region of the column to be treated, for example: lumbar sacral, thoracolombar; cervicothoracic. This is why the device of the present invention does not comprise an upper or lower region, because such guidelines will only be defined during the surgical treatment of implanting the device in the patient.

Additionally, the arrangement of the hollow tubular section of the central body (1) is designed to enhance dynamization of the device. More preferably, a micro-dynamization needed for surgical applications in the vertebral column. In other words, said opening in the central body enables the device to have micro-movements to adjust to movements of the column, such that the device of the present invention has a semi-rigid structure.

The opening (4) of the central body (1) may comprise different formats, for example: circular, rectangular, oblong, oval, provided that an area with a width (a) equal or greater than the width (b) of the wall of the central body (1) is maintained. Preferably, the ratio between widths (a) and (b) is between 1:1 and 3:1, and said ratio depends on the level of dynamization desired.

It is also important to emphasize that the dimensions of the device may undergo alterations to meet the needs of the anatomy of the patient's bones. More particularly, as persons skilled in the art are no doubt aware, bone structures may vary in size and particularities depending on age, size and characteristics of the individual.

Accordingly, some patients may require devices with a larger or smaller central body, larger or smaller pairs of wings, and it is also possible that patients may require that the device comprise one pair of wings that is larger than the other.

Therefore, in view of the above, it is clear that the interspinous device, according to the present invention, comprises characteristics that provide various benefits and technical-functional effects that enhance safety, efficiency and agility in surgical processes and treatments, and reduces the sequelae sustained by the patients. Besides comprising a unique structure in a material that is easy to manufacture, when compared with the state of the art.

1. INTERSPINOUS DEVICE, that comprises a central body (1) having two pairs of wings (2), characterized in that each pair of wings (2) comprises at least one anchoring through hole (3), and said central body (1) comprises a closed tubular section having a central opening (4).

2. INTERSPINOUS DEVICE, according to claim 1, characterized by being made of a polymeric material.

3. INTERSPINOUS DEVICE, according to claim 2, characterized in that the polymeric material is PEEK—poly (ether-ether-ketone) kind.

4. INTERSPINOUS DEVICE, according to claim 2, characterized in that the polymeric material is of the PAEK, PEEKK, PEKK and PEK kind.

5. INTERSPINOUS DEVICE, according to claim 1, characterized in that said central body (1) comprises a flattened, oblong shape.

6. INTERSPINOUS DEVICE, according to claim 1, characterized in that said central body (1) comprises at least a bump (5) adjacent to each pair of wings (2).

7. INTERSPINOUS DEVICE, according to claim 6, characterized in that said bumps (5) comprise a pointed triangle format, of the clamp kind.

8. INTERSPINOUS DEVICE, according to claim 1, characterized in that the inner surfaces of said wings (2) have protrusions (6).

9. INTERSPINOUS DEVICE, according to claim 1, characterized in that said protrusions are partially or wholly distributed on the inner surface of said wings (2).

10. INTERSPINOUS DEVICE, according to claim 1, characterized in that the anchoring holes (3) receive sutures, metallic cerclages, screws or any other equivalent means known in the field of surgery.

11. INTERSPINOUS DEVICE, according to claim 1, characterized in that the anchoring holes (3) comprise circular, rectangular, triangular, or oval formats, or a combination thereof.

12. INTERSPINOUS DEVICE, according to claim 1, characterized in that said central body (1) further comprises small canals (7) for accommodating metallic pins.

13. INTERSPINOUS DEVICE, according to claim 12, characterized in that said canals (7) are rectilinear and perpendicular to the central body (1).

14. INTERSPINOUS DEVICE, according to claim 1, characterized in that the rear portion of said central body comprises at least one hole (8) for receiving surgical instruments.

15. INTERSPINOUS DEVICE, according to claim 13, characterized in that said at least one hole (8) comprises a grooved surface.

16. INTERSPINOUS DEVICE, according to claim 1, characterized in that the pairs of wings (2) are arranged in skewed fashion in relation to each other.

17. INTERSPINOUS DEVICE, according to claim 16, characterized in that at least one of said pairs of wings (2) is arranged in relation to the center of the central body (1).

18. INTERSPINOUS DEVICE, according to claim 16, characterized in that at least one of said pairs of wings (2) is arranged relatively aligned with the rear portion of the central body (1).

19. INTERSPINOUS DEVICE, according to claim 1, characterized in that an opening (4) of said central body (1) comprises a circular, rectangular, oval or oblong format.

20. INTERSPINOUS DEVICE, according to claim 19, characterized in that said opening (4) of the central body (1) comprises an area with a width (a) equal to or larger than the width (b) of the wall of the central body (1), pursuant to the dynamization desired.

21. INTERSPINOUS DEVICE, according to claim 20, characterized in that a ratio between widths (a) and (b) is between 1:1 and 3:1.

22. INTERSPINOUS DEVICE, according to claim 1, characterized by being applied in column treatments, in the lumbar sacral, thoracolombar and cervicothoracic regions.
